(19) United States
(12) Patent Application Publication
(10) Pub. No.: US 2005/0203772 A1
(43) Pub. Date: Sep. 15, 2005

(54) Examination appointment method and examination appointment system

(75) Inventor: Takeshi Funahashi, Kaisei-machi (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/068,966

(22) Filed: Mar. 2, 2005

(30) Foreign Application Priority Data
Mar. 11, 2004 (JP) 2004-069133

(51) Int. Cl. G06F 17/60

(52) U.S. Cl. 705/2

(57) ABSTRACT

An examination appointment method capable of reducing a burden for printing out hospital information and map images of an examination appointment destination. The method is used in an examination appointment system which is configured by connecting a client terminal and a server provided in a data center via a network and making an appointment of an examination intended for diagnosis. The method includes the steps of: transmitting hospital information data and/or map image data on a hospital, which is an appointment destination of the examination, from the server to the client terminal when requested by the client terminal; transmitting examination appointment information data, which represents examination appointment information on the appointment of the examination, from the client terminal to the server; printing out an image based on the hospital information data and/or the map image data by background processing in the client terminal; and receiving the examination appointment information in the server.

### BACKGROUND OF THE INVENTION

1. Field of the Invention

The present invention relates to an examination appointment method and an examination appointment system for making an appointment of an examination intended for diagnosis of a patient via a network between medical facilities.

2. Description of a Related Art

Conventionally, in medical facilities such as hospitals, examinations using radiation (X-rays or the like), ultrasonic waves, MR (magnetic resonance), etc. have been performed. However, there may be cases where there is no specialized doctor or examination equipment in small hospitals or clinics and sometimes disorders of patients can not be coped with. In such a case, doctors of small hospitals or clinics (introduction source medical institutions) sometimes introduce patients to hospitals having specialized doctors or hospitals having medical equipment (introduction destination medical institutions). A medical information system for introducing patients via a network is known (see JP-A-2002-73822, for example).

In a medical information system disclosed in JP-A-2002-73822, when a consultation appointment is set, geographical information of an introduction destination medical institution and precaution information are transmitted from a processing unit of a medial information center to a processing unit of an introduction source medical institution. However, in this medical information system, a doctor or the like of the introduction source medical institution must provide an instruction to print out to the processing unit in order to print out the geographical information of the introduction destination medical institution and the precaution information, and that becomes a burden on the doctor or the like.

### SUMMARY OF THE INVENTION

Accordingly, in view of the above-described problems, an object of the present invention is to provide an examination appointment method and an examination appointment system which are capable of reducing a burden on a doctor or the like for printing out hospital information and/or map images of an introduction destination medical institution as an examination appointment destination.

In order to solve the above-described problems, an examination appointment method according to the present invention is an examination appointment method used in an examination appointment system which is configured by connecting a client terminal and a server provided in a data center via a network and making an appointment of an examination intended for diagnosis, the method comprising the steps of: (a) transmitting hospital information data and/or map image data on a hospital, which is an appointment destination of the examination, from the server to the client terminal when requested by the client terminal; (b) transmitting examination appointment information data, which represents examination appointment information on the appointment of the examination, from the client terminal to the server; (c) printing out an image based on the hospital information data and/or the map image data by background processing in the client terminal; and (d) receiving the examination appointment information in the server.

Further, an examination appointment system according to the present invention is configured by connecting a client terminal and a server provided in a data center via a network, and making an appointment of an examination intended for diagnosis, wherein: the server includes: a recording medium for recording examination appointment information data on the appointment of the examination, and hospital information data and/or map image data on a hospital which is an appointment destination of the examination; a hospital information transmission processing unit for transmitting the hospital information data and/or the map image data on the hospital, which is the appointment destination of the examination, to the client terminal when requested by the client terminal; and an examination appointment information reception processing unit for receiving the examination appointment information data on the examination appointment from the client terminal, and recording the examination appointment information data in the recording medium, and the client terminal includes: an examination appointment information input processing unit for receiving input of the examination appointment information data from a user, and transmitting the examination appointment information data to the server; and a hospital information printing processing unit for printing out an image based on the hospital information data and/or the map image data by background processing.

According to the examination appointment method and the examination appointment system, since the hospital information and/or the map image of the examination appointment destination can be printed out by the background processing in the client terminal of the examination appointment source, the burden on the doctor or the like for printing out the hospital information and/or the map image of the examination appointment destination can be reduced.

### BRIEF DESCRIPTION OF THE DRAWINGS

FIG. 1 is a block diagram showing the configuration of an examination appointment system for realizing an examination appointment method according to one embodiment of the present invention;

FIG. 2 is a block diagram showing the internal constitution of an examination appointment server 2 shown in FIG. 1;

FIG. 3 shows the relationship between hospital information recorded in a hospital information recording unit 31 and hospital map image data recorded in a hospital map image recording unit 32 in FIG. 2;

FIG. 4 is a block diagram showing the internal constitution of a client terminal 3 in FIG. 1;

FIG. 5 shows the hardware configuration of the client terminal 3 in FIG. 1;

FIG. 6 is a flowchart showing the operation of the client terminal 3 and the examination appointment server 2 in FIG. 1;

FIG. 7 shows an example of an image displayed in the client terminal 3 in FIG. 1; and

FIG. 8 is a flowchart showing the operation of the client terminal 3 and the examination appointment server 2 in FIG. 1.

### DESCRIPTION OF THE PREFERRED EMBODIMENTS

Hereinafter, best modes of the present invention will be described in detail by referring to the drawings. The same component elements are assigned with the same reference numerals and the description thereof will be omitted.

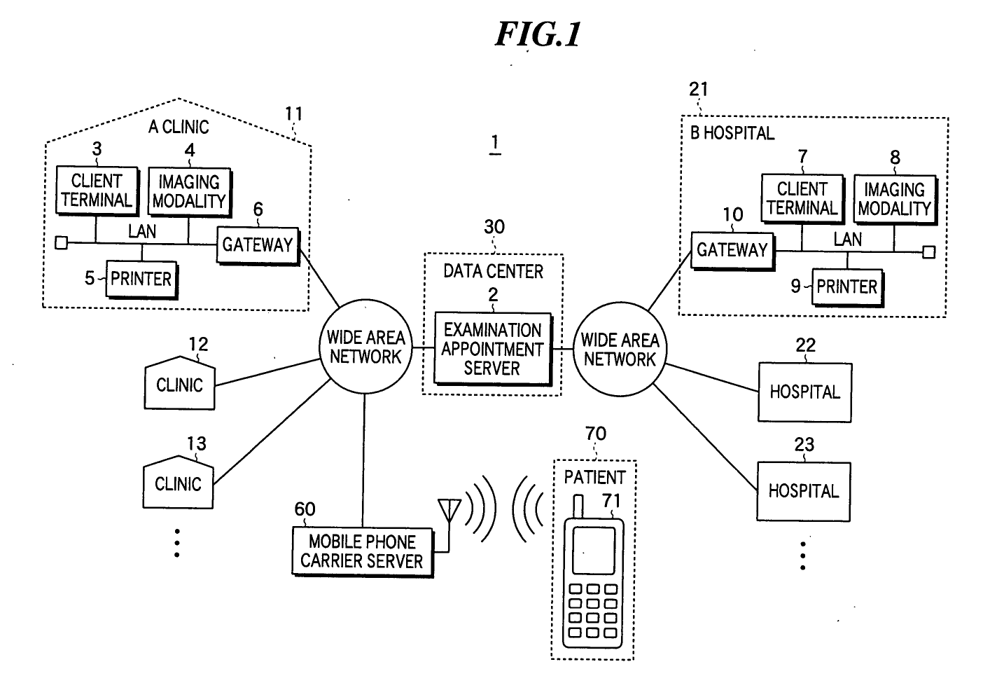

FIG. 1 is a block diagram showing the configuration of an examination appointment system for realizing an examination appointment method according to one embodiment of the present invention. As shown in FIG. 1, an examination appointment system 1 is configured by connecting client terminals 3, which are provided in clinics 11, 12, 13, . . . as examples of medical facilities, and client terminals 7, which are provided in hospitals 21, 22, 23, . . . as examples of medical facilities, to an examination appointment server 2, which is provided in a data center 30, via a wide area network, respectively. Further, a mobile phone carrier server 60 is connected to the wide area network, and this mobile phone carrier server 60 can wirelessly communicate with a mobile phone device 71 of a patient 70.

The examination appointment system 1 is a system for making appointments of examinations in the hospitals 21, 22, 23, . . . from the client terminals 3 of the clinics 11, 12, 13, . . . . As the wide area network, a dedicated line, a public line, the Internet, or the like can be utilized.

In each of the clinics 11, 12, 13, . . . , an imaging modality 4 such as a radiation imaging apparatus or an ultrasonic diagnosing apparatus, the client terminal 3 for inputting medical image data from the imaging modality 4 and for making examination appointments to the hospitals 21, 22, 23, . . . , or the like, a gateway 6 having a firewall function, a printer 5 for printing out medical images on films or the like, etc. are connected via a local area network (LAN).

The client terminal 3 can control the imaging modality 4, input image data from the imaging modality 4, and further, perform image processing on image data and display images for diagnosis based on the image data.

In each of the hospitals 21, 22, 23, . . . , an imaging modality 8 such as a CT (computed tomography) apparatus and an MRI (Magnetic Resonance Imaging) apparatus, the client terminal 7 for inputting medical image data from the imaging modality 8, a gateway 10 having a firewall function, a printer 9 for printing out medical images on films or the like, etc. are connected via a local area network (LAN).

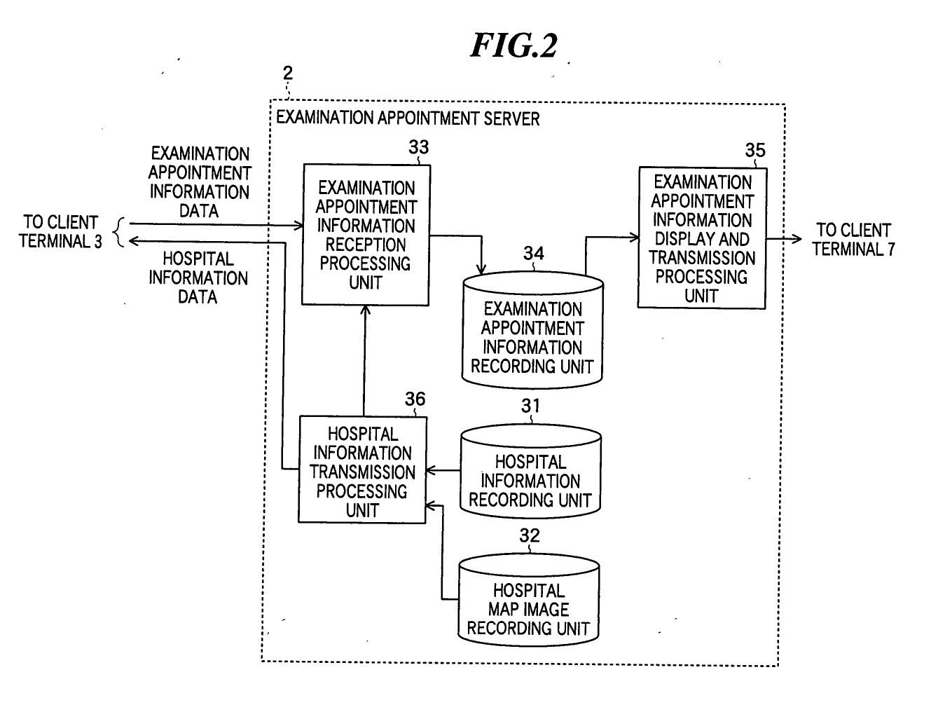

FIG. 2 is a block diagram showing an outline of the configuration of the examination appointment server 2 installed in the data center 30. As shown in FIG. 2, the examination appointment server 2 includes a hospital information recording unit 31, a hospital map image recording unit 32, an examination appointment information reception processing unit 33, an examination appointment data recording unit 34, an examination appointment information display and transmission processing unit 35, and a hospital information transmission processing unit 36.

The hospital information recording unit 31 has recorded plural hospital information data representing hospital information on the hospitals 21, 22, 23, . . . , respectively. The hospital information includes items such as a name of a hospital, an address of a hospital, a telephone number of a hospital, and cautions as to examination. These items are just examples and not all of the items are essential, and other items may be included.

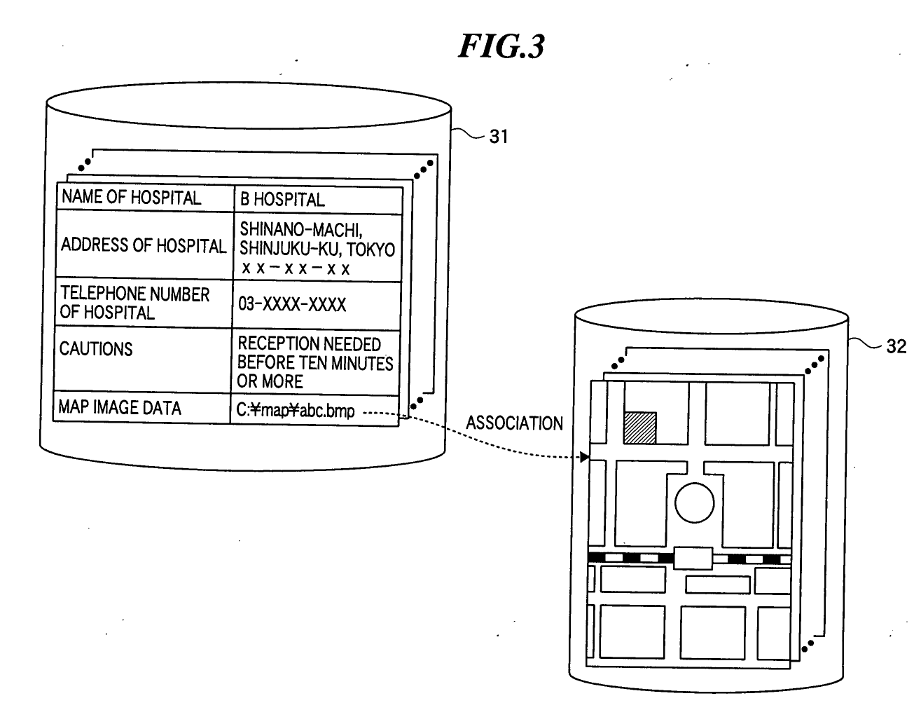

The hospital map image recording unit 32 has recorded plural map image data representing maps of the hospitals 21, 22, 23, . . . , respectively, and the plural map image data are associated with plural hospital information data recorded in the hospital information recording unit 31. Specifically, as shown in FIG. 3, a directory path and a file name of the map image data of the hospital of interest is added to the hospital information. By the way, a URL may be added to the hospital information in place of the directory path and the file name, or other identifiers, pointers, or the like for identifying map image data may be added thereto.

Referring to FIG. 2 again, the hospital information transmission processing unit 36 reads out hospital information data on an examination appointment destination hospital selected by the doctor or the like of the examination appointment source from the hospital information recording unit 31, and reads out map image data associated with the read hospital information data from the hospital map image recording unit 32 based on the directory path and the file name added to the hospital information data, and sends the data to the client terminal 3. Further, the hospital information transmission processing unit 36 sends the name of the examination appointment destination hospital selected by the doctor or the like of the examination appointment source to the examination appointment information reception processing unit 33.

The examination appointment information reception processing unit 33 receives examination appointment information from the client terminal 3 of the examination appointment destination, checks as to there is no overlapping appointment, and then, allows the examination appointment information recording unit 34 to record examination appointment information data representing the received examination appointment information.

When an examination appointment is made, the examination appointment information display and transmission processing unit 35 transmits that effect with an e-mail to the client terminal 7 of the examination appointment destination hospital. By the way, the examination appointment information display and transmission processing unit 35 may transmit the examination appointment information data, which is recorded in the examination appointment information recording unit 34, to the client terminal 7 of the examination appointment destination hospital, and display the examination appointment information.

The examination appointment information reception processing unit 33, the examination appointment information display and transmission processing unit 35, and the hospital information transmission processing unit 36 shown in FIG. 2 can be configured by a CPU (central processing unit) and software (program). This program, the hospital information data, the hospital map image data, and the examination appointment information data can be recorded in a recording medium such as a hard disk, a flexible disk, an MO, an MT, a RAM, a CD-RW, or a DVD-RAM.

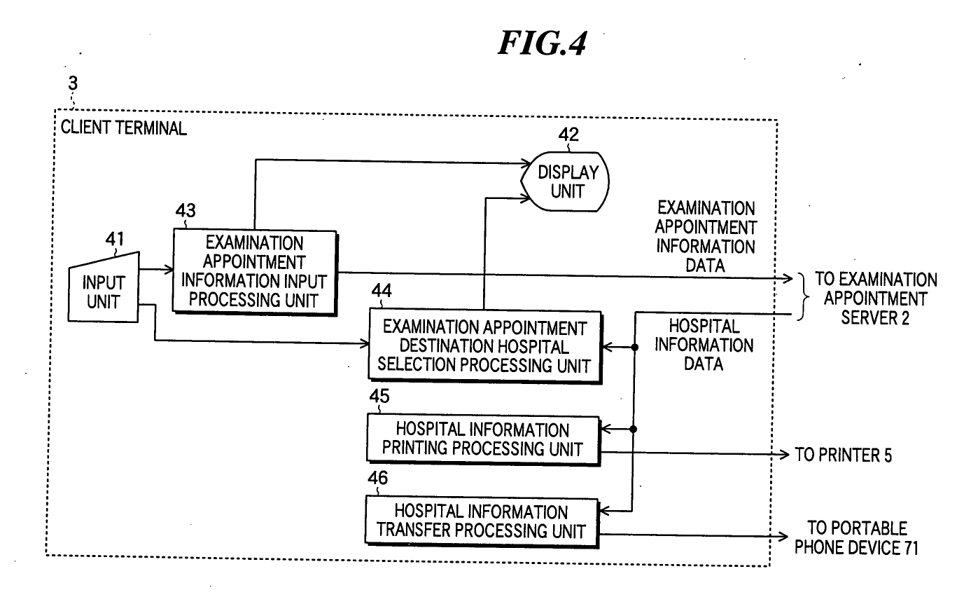

FIG. 4 is a block diagram showing the constitution of the client terminal 3. As shown in FIG. 4, the client terminal 3 includes an input unit 41, a display unit 42, an examination appointment information input processing unit 43, an examination appointment destination hospital selection processing unit 44, a hospital information printing processing unit 45, and a hospital information transfer processing unit 46.

The input unit 41 is a keyboard, a mouse, or the like by which a doctor or the like of the examination appointment source inputs examination appointment information on examination appointments or the like, and the display unit 42 is a CRT, an LCD, or the like for displaying characters, images, etc.

The examination appointment information input processing unit 43 displays the examination appointment information, which is inputted by the doctor or the like of the examination appointment source to the input unit 41, on the display unit 42, and transmits examination appointment information data representing the examination appointment information to the examination appointment server 2.

The examination appointment destination hospital selection processing unit 44 receives hospital information data and map image data of the examination appointment destination hospital, which is selected by the doctor or the like of the examination appointment source, from the examination appointment server 2, and displays the hospital information and the map image of the examination appointment destination hospital on the display unit 42.

The hospital information printing processing unit 45 receives the hospital information data and the map image data of the examination appointment destination hospital from the examination appointment server 2, and allows the printer 5 to print the hospital information and the map image of the examination appointment destination hospital by background processing.

The hospital information transfer processing unit 46 receives the hospital information data and the map image data of the examination appointment destination hospital from the examination appointment server 2, and transfers these data to the portable phone device 71 of the target patient of examination by background processing.

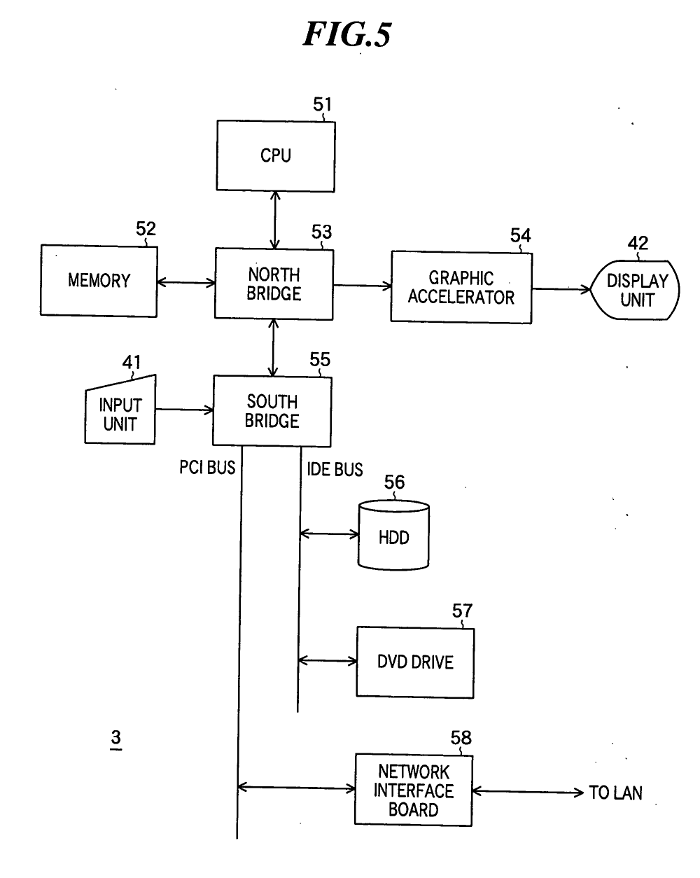

The client terminal 3 has a hardware configuration as shown in FIG. 5. As shown in FIG. 5, a CPU (central processing unit) 51, a memory 52, and a graphic accelerator 54 are connected via a north bridge 53, and the graphic accelerator 54 is connected to the display unit 42.

Further, a south bridge 55 is connected to the north bridge 53, and the input unit 41 is connected to the south bridge 55. The south bridge 55 has an IDE (integrated device electronics) bus and a PCI (peripheral component interconnect) bus, and an HDD (hard disc drive) 56 and a DVD (digital versatile disc) drive 57 are connected to the IDE bus. A network interface board 58 is connected to the PCI bus, and the client terminal 3 is connected to a LAN within a clinic by the network interface board 58.

In the HDD 56, an operating system program capable of multitask processing and various kinds of device drivers are recorded, and the CPU 51 executes these programs so as to control the entire client terminal 3.

Further, in the HDD 56, plural application programs for respectively realizing examination appointment information input processing, examination appointment destination hospital selection processing, hospital information printing processing, and hospital information transfer processing are also recorded, and the CPU 51 executes the plural application programs as multitask. Thereby, the operation of the examination appointment information input processing unit 43, the examination appointment destination hospital selection processing unit 44, the hospital information printing processing unit 45, and the hospital information transfer processing unit 46 shown in FIG. 4 is realized.

When the CPU 51 executes the above-described various kinds of programs, the HDD 56 and the memory 52 are respectively used as work areas.

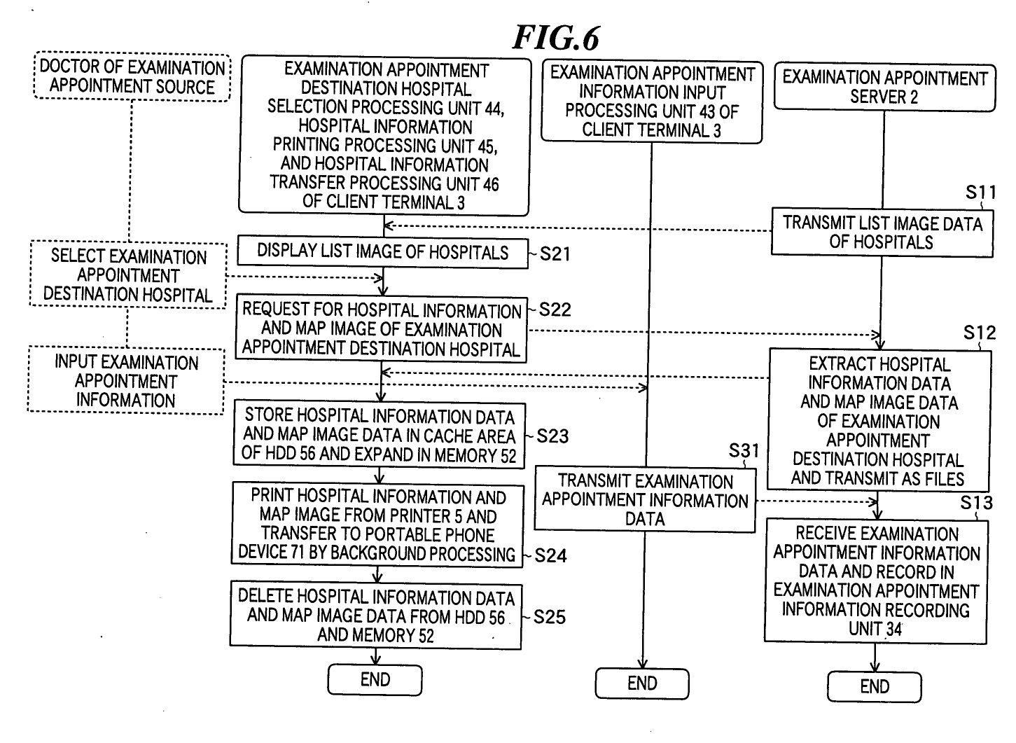

As below, the first examination appointment operation of the examination appointment system 1 will be described by referring to a flowchart shown in FIG. 6. Here, a doctor of an A clinic 11 (see FIG. 1) makes an appointment of an examination using the imaging modality 8 of a B hospital 21 (see FIG. 1).

First, the hospital information transmission processing unit 36 of the examination appointment server 2 transmits list image data for displaying a list image of the hospitals 21, 22, 23, . . . , to the examination appointment destination hospital selection processing unit 44 of the client terminal 3 (step S11).

The examination appointment destination hospital selection processing unit 44 of the client terminal 3 displays the list image of the hospitals 21, 22, 23, . . . based on the list image data transmitted from the examination appointment server 2 (step S21).

The doctor or the like of the examination appointment source selects the B hospital 21, which is a desired hospital, from the listed hospitals as an examination appointment destination hospital.

The examination appointment destination hospital selection processing unit 44 of the client terminal 3 issues a request to transmit the hospital information and the map image of the B hospital 21, which is selected as the examination appointment destination hospital, to the examination appointment server 2 (step S22).

The hospital information transmission processing unit 36 of the examination appointment server 2 extracts the hospital information data of the B hospital 21, which is the examination appointment destination hospital, from the hospital information recording unit 31, extracts the map image data of the B hospital 21 from the hospital map image recording unit 32, and transmits the extracted hospital information data and the extracted map image data as files to the client terminal 3 (step S12). The hospital information data and the map image data of the B hospital 21 transmitted as files from the examination appointment server 2 are stored in a cache area within the HDD 56 (see FIG. 5) of the client terminal 3, and expanded in the memory 52 (see FIG. 5) (step S23).

On the other hand, simultaneously, the doctor or the like of the A clinic 11 which is the examination appointment source inputs examination appointment information on examination appointments (e.g., name of patient, date of birth, sex, patient's card number, name of hospital of examination appointment destination, name of doctor of examination appointment destination, type of examination appointment, name of hospital of examination appointment destination, date and time of examination appointment, etc.) to the examination appointment information input processing unit 43 of the client terminal 3. The above-described items are just examples and not essential, and other items may be inputted.

In the client terminal 3, the examination appointment information input processing program, the examination appointment destination hospital selection processing program, the hospital information printing processing program, and the hospital information transfer processing program are executed as multitask, and the doctor or the like of the A clinic 11 can input the examination appointment information regardless of the execution statuses of the examination appointment destination hospital selection processing program, the hospital information printing processing program, and the hospital information transfer processing program.

The examination appointment information input processing unit 43 of the client terminal 3 transmits examination appointment information data, which represents the examination appointment information inputted by the doctor or the like of the A clinic 11, to the examination appointment information reception processing unit 33 of the examination appointment server 2 (step S31).

The examination appointment information reception processing unit 33 of the examination appointment server 2 searches the examination appointment information recording unit 34 according to the received examination appointment information data, checks whether or not there is an examination appointment that overlaps in the period, and then, allows the examination appointment information recording unit 34 to record the received examination appointment information data if there is no overlapping examination appointment (step S13). In addition, when the examination appointment information data is recorded in the examination appointment information recording unit 34, the examination appointment information display and transmission processing unit 35 (see FIG. 2) may transmit an e-mail for notifying that the examination appointment has been made, to the client terminal 7 provided in the B hospital 21.

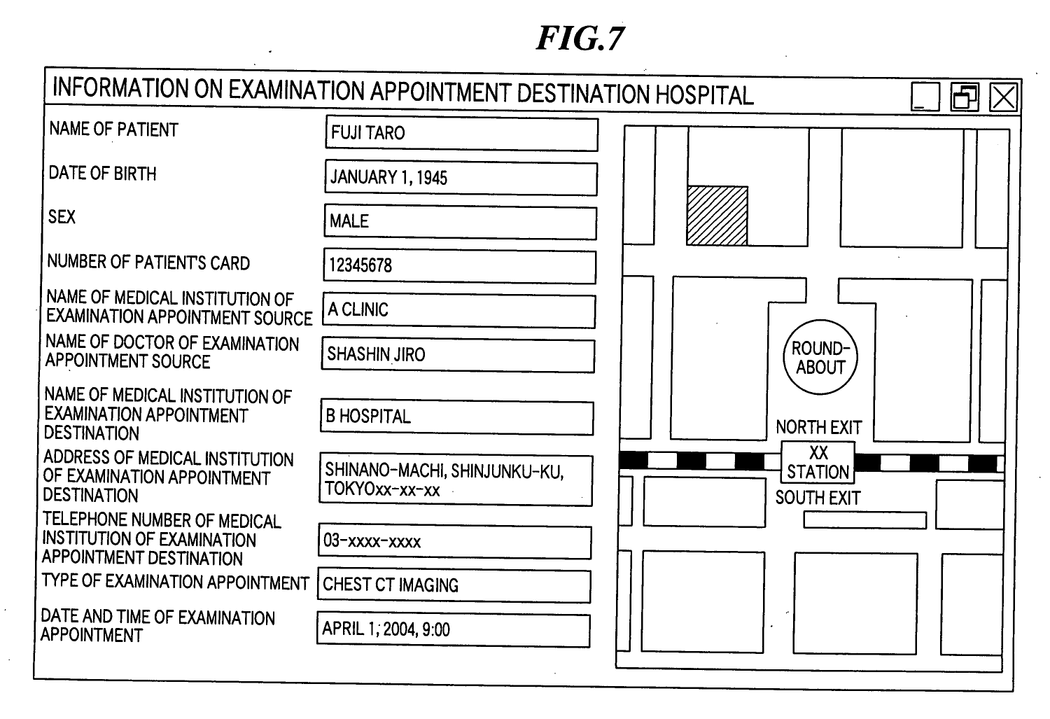

On the other hand, the examination appointment destination hospital selection processing unit 44 of the client terminal 3 provided in the A clinic 11 allows the display unit 42 to display an image based on the hospital information data and the map image data of the B hospital 21 which are recorded in the cache area within the HDD 56 and the memory 52. FIG. 7 shows an example of display screen at this time. By the way, as shown in FIG. 7, the entire or a part of the examination appointment information may be displayed with the hospital information and the map image of the B hospital 21.

Then, the hospital information printing processing unit 45 of the client terminal 3 prints out the hospital information and the map image of the B hospital 21 (the image based on the hospital information data and the map image data of the B hospital 21) from the printer 5 by background processing. Similarly, the hospital information transfer processing unit 46 of the client terminal 3 transfers the hospital information data and the map image data of the B hospital 21 to the portable phone device 71 of the patient by background processing (step S24). By the way, the hospital information transfer processing unit 46 may transfer the hospital information data and the map image data of the B hospital 21 not to the portable phone device 71 of the patient, but to a facsimile machine of the patient.

Subsequently, in view of securing of the areas of the HDD 56 and/or the memory 52, the client terminal 3 deletes the hospital information data and the map image data of the B hospital 21 which are recorded in the cache area within the HDD 56 and/or the memory 52 (step S25).

As described above, according to the embodiment, since the hospital information and the map image of the B hospital 21 which is the examination appointment destination hospital is printed out by background processing, it is no longer necessary for the doctor or the like in charge of the examination appointment to provide an instruction for printing out, and the burden on the doctor or the like can be reduced. Further, since the doctor or the like can input the examination appointment information while the hospital information and the map image of the B hospital 21, which is the examination appointment destination hospital, is printed out by background processing, the time for the appointment work can be shortened. Furthermore, since the hospital information data and the map image data of the B hospital 21 which is the examination appointment destination hospital is transferred to the terminal (e.g., the portable phone device 71, the facsimile machine, or the like) of the patient who is the target of examination, by background processing, that provides convenience for the patient.

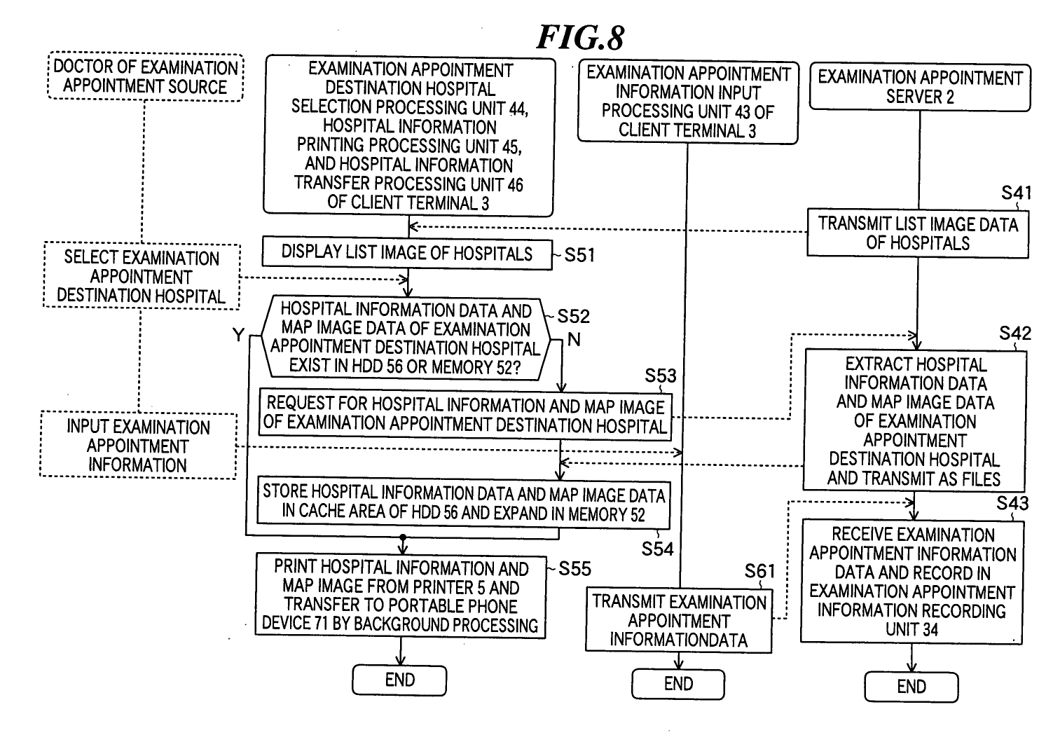

Next, the second examination appointment operation of the examination appointment system 1 will be described by referring to a flowchart shown in FIG. 8. In the second examination appointment operation, as well as in the above-described first examination appointment operation, the doctor of the A clinic 11 (see FIG. 1) makes the appointment of the examination using the imaging modality 8 of the B hospital 21 (see FIG. 1).

In the above-described first examination appointment operation, after the printing out of the hospital information and the map image and the transfer of the hospital information data and the map image data (step S24 in FIG. 6) are completed, the hospital information data and the map image data of the B hospital 21, which are recorded in the cache area within the HDD 56 and the memory 52, are deleted (step S25 in FIG. 6). On the other hand, in the second examination appointment operation, even after the printing out of the hospital information and the map image and the transfer of the hospital information data and the map image data are completed, the hospital information data and the map image data of the B hospital 21 which are recorded in the cache area within the HDD 56 and memory 52 are not deleted, but reused.

In the second examination appointment operation, first, the hospital information transmission processing unit 36 of the examination appointment server 2 transmits the list image data for displaying the list image of the hospitals 21, 22, 23, . . . to the examination appointment destination hospital selection processing unit 44 of the client terminal 3 (step S41).

The examination appointment destination hospital selection processing unit 44 of the client terminal 3 displays the list image of the hospitals 21, 22, 23, . . . based on the list image data transmitted from the examination appointment server 2 (step S51).

The doctor or the like of the examination appointment source selects the B hospital 21, which is the desired hospital, from the listed hospitals as an examination appointment destination hospital.

The examination appointment destination hospital selection processing unit 44 of the client terminal 3 checks whether or not the hospital information data and the map image data of the B hospital 21 which is selected as the examination appointment destination hospital, have been previously transmitted from the examination appointment server 2 and they exist in the cache area within the HDD 56 or the memory 52. If they exist, the process moves to step S55, and, if not, the process moves to step S53 (step S52).

In the case where the hospital information data and the map image data of the B hospital 21 which is selected as the examination appointment destination hospital, do not exist in the cache area within the HDD 56 or the memory 52, the examination appointment destination hospital selection processing unit 44 of the client terminal 3 issues a request of the hospital information and the map image of the B hospital 21 to the examination appointment server 2 (step S53).

When receiving the request from the client terminal 3, the hospital information transmission processing unit 36 of the examination appointment server 2 extracts the hospital information data of the B hospital 21 from the hospital information recording unit 31, extracts the map image data of the B hospital 21 from the hospital map image recording unit 32, and transmits the extracted hospital information data and the extracted map image data to the client terminal 3 (step S42). The hospital information data and the map image data of the B hospital 21 transmitted as files from the examination appointment server 2 are stored in the cache area within the HDD 56 (see FIG. 5) of the client terminal 3 and expanded in the memory 52 (see FIG. 5) (step S54).

Simultaneously, the doctor or the like of the A clinic 11 which is the examination appointment source inputs the examination appointment information on the examination appointments to the examination appointment information input processing unit 43 of the client terminal 3.

In the client terminal 3, the examination appointment information input processing program, the examination appointment destination hospital selection processing program, the hospital information printing processing program, and the hospital information transfer processing program are executed as multitask, and the doctor or the like of the A clinic 11 can input the examination appointment information regardless of the execution statuses of the examination appointment destination hospital selection processing program, the hospital information printing processing program, and the hospital information transfer processing program.

The examination appointment information input processing unit 43 of the client terminal 3 transmits examination appointment information data representing the examination appointment information, which is inputted by the doctor or the like of the A clinic 11, to the examination appointment information reception processing unit 33 of the examination appointment server 2 (step S61).

The examination appointment information reception processing unit 33 of the examination appointment server 2 searches the examination appointment information recording unit 34 according to the examination appointment information data, checks whether or not there is the examination appointment that overlaps in the period, and then, allows the examination appointment information recording unit 34 to record the received examination appointment information data if there is no overlapping examination appointment (step S43).

On the other hand, the examination appointment destination hospital selection processing unit 44 of the client terminal 3 allows the display unit 42 to display the image based on the hospital information data and the map image data of the B hospital 21 which are recorded in the cache area within the HDD 56 and the memory 52. Then, the hospital information printing processing unit 45 of the client terminal 3 prints out the hospital information and the map image of the B hospital 21 from the printer 5 by background processing. Similarly, the hospital information transfer processing unit 46 of the client terminal 3 transfers the hospital information data and the map image data of the B hospital 21 to the portable phone device 71 of the patient by background processing (step S55).

Note that, in view of the traffic reduction on the network and the improvement in the processing speed, the client terminal 3 do not delete the hospital information data and the map image data of the B hospital 21, which are recorded in the cache area within the HDD 56 and the memory 52, in order to use them (see steps S52 to S54). Even in this case, when there is no free space in the cache area within the HDD 56 and/or the memory 52 or after a predetermined time has been elapsed from the time when they are written in the cache area within the HDD 56 and/or the memory 52, the hospital information data and/or the map image data of the B hospital 21 may be deleted appropriately.

As described above, according to the second examination appointment operation, when the hospital information data and the map image data of the examination appointment destination hospital have been recorded in the cache area within the HDD 56 and the memory 52, the transmission of the hospital information data and the map image data from the examination appointment server 2 to the client terminal 3 is not performed, and thereby, the traffic reduction on the network, the improvement in the processing speed, etc. can be performed.

In addition, in the embodiment, the client terminal 3 records the hospital information data and the map image data in the cache are a with in the HDD 56 and the memory 52. However, instead of the hospital information data and the map image data, the directory path and the file name of the hospital information data and the map image data in the hospital information recording unit 31 and the hospital map image recording unit 32 within the examination appointment server 2 may be recorded, and the directory path and the file name may be designated when the hospital information and the map image are requested to the examination appointment server 2.

Further, the image based on the hospital information data and the map image data of the examination appointment destination hospital (the hospital information and the map image of the examination appointment destination hospital) is printed out. However, an image based on the hospital information data or the map image data of the examination appointment destination hospital may be printed out.

1. An examination appointment method used in an examination appointment system which is configured by connecting a client terminal and a server provided in a data center via a network and making an appointment of an examination intended for diagnosis, said method comprising the steps of:
(a) transmitting hospital information data and/or map image data on a hospital, which is an appointment destination of the examination, from said server to said client terminal when requested by said client terminal;
(b) transmitting examination appointment information data, which represents examination appointment information on the appointment of the examination, from said client terminal to said server;
(c) printing out an image based on said hospital information data and/or said map image data by background processing in said client terminal; and
(d) receiving said examination appointment information in said server.

2. The examination appointment method according to claim 1, further comprising the steps of:
(e) transmitting image data for displaying at least one candidate hospital, which is the appointment destination of the examination, from said server to said client terminal; and
(f) transmitting hospital selection information data on hospital selection from said client terminal to said server,
wherein step (a) includes transmitting said hospital information data and/or said map image data on the selected hospital from said server to said client terminal.

3. The examination appointment method according to claim 1, wherein step (c) includes caching said hospital information data and/or said map image data, which are transmitted from said server, in a recording medium, and printing out an image based on said hospital information data and/or said map image data, which are cached in said recording medium, by background processing in said client terminal.

4. The examination appointment method according to claim 3, wherein said recording medium includes a memory and/or a hard disk drive.

5. The examination appointment method according to claim 1, further comprising the step of:
(g) transferring said hospital information data and/or said map image data, which are cached in said recording medium, by background processing from said client terminal to a terminal of a patient who is a target of said examination.

6. The examination appointment method according to claim 5, said terminal includes a portable phone device and/or a facsimile machine.

7. The examination appointment method according to claim 1, further comprising the step of:
(h) deleting said hospital information data and/or said map image data, which are cached in said recording medium, in said client terminal after step (c).

8. The examination appointment method according to claim 5, further comprising the step of:
(h′) deleting said hospital information data and/or said map image data, which are cached in said recording medium, in said client terminal after step (g).

9. The examination appointment method according to claim 1, further comprising the step of:
(i) requesting for transmission of said hospital information data and/or said map image data to said server in said client terminal when said hospital information data and/or said map image data is not cached in said recording medium,
wherein step (a) includes transmitting said hospital information data and/or said map image data from said server to said client terminal when requested by said client terminal at step (i).

10. The examination appointment method according to claim 9, further comprising the step of:
(j) caching a directory path and/or a file name of said hospital information data and/or said map image data within said server, in said recording medium in said client terminal,
wherein step (i) includes designating said directory path and/or said file name and requesting for transmission of said hospital information data and/or said map image data to said server in said client terminal when said hospital information data and/or said map image data is not cached in said recording medium.

11. An examination appointment system configured by connecting a client terminal and a server provided in a data center via a network, and making an appointment of an examination intended for diagnosis, wherein:
said server includes:
a recording medium for recording examination appointment information data on the appointment of the examination, and hospital information data and/or map image data on a hospital which is an appointment destination of the examination;
a hospital information transmission processing unit for transmitting the hospital information data and/or the map image data on the hospital, which is the appointment destination of the examination, to said client terminal when requested by said client terminal; and
an examination appointment information reception processing unit for receiving the examination appointment information data on the examination appointment from said client terminal, and recording the examination appointment information data in said recording medium, and
said client terminal includes:
an examination appointment information input processing unit for receiving input of said examination appointment information data from a user, and transmitting the examination appointment information data to said server; and
a hospital information printing processing unit for printing out an image based on said hospital information data and/or said map image data by background processing.

12. The examination appointment system according to claim 11, wherein said hospital information transmission processing unit of said server transmits image data for displaying at least one candidate hospital, which is the appointment destination of the examination, to said client terminal, and transmits said hospital information data and/or said map image data on a hospital selected based on hospital selection information data on hospital selection, which is transmitted from said client terminal, to said client terminal.

13. The examination appointment system according to claim 11, wherein said client terminal caches said hospital information data and/or said map image data, which are transmitted from said server, in a recording medium, and prints out an image based on said hospital information data and/or said map image data, which are cached in said recording medium, by background processing.

14. The examination appointment system according to claim 13, wherein said recording medium includes a memory and/or a hard disk drive.

15. The examination appointment system according to claim 11, wherein said client terminal further includes a hospital information transfer processing unit for transferring said hospital information data and/or said map image data, which are cached in said recording medium, by background processing to a terminal of a patient who is a target of said examination.

16. The examination appointment system according to claim 15, wherein said terminal includes a portable phone device and/or a facsimile machine.

17. The examination appointment system according to claim 11, wherein said client terminal deletes said hospital information data and/or said map image data, which are cached in said recording medium, after printing out of an image based on said hospital information data and/or said map image data and/or transfer of said hospital information data and/or said map image data to said terminal.

18. The examination appointment system according to claim 11, wherein:
said examination appointment destination hospital selection processing unit of said client terminal requests for transmission of said hospital information data and/or said map image data to said server when said hospital information data and/or said map image data is not cached in said recording medium, and
said hospital information transmission processing unit of said server transmits said hospital information data and/or said map image data to said client terminal when requested by said client terminal.

19. The examination appointment system according to claim 18, wherein said client terminal caches a directory path and/or a file name of said hospital information data and/or said map image data within said server, in said recording medium, and designates said directory path and/or said file name and requests for transmission of said hospital information data and/or said map image data to said server when said hospital information data and/or said map image data is not cached in said recording medium.
